(19) United States
(12) Patent Application Publication
(10) Pub. No.: US 2026/0083929 A1
(43) Pub. Date: Mar. 26, 2026

(54) Medical Gas Retention system

(71) Applicant: OXYGEN RETENTION SYSTEMS, Lake Forest, IL (US)

(72) Inventors: Todd Arndt, Lake Forest, IL (US); Trent Swarthout, Lake Forest, IL (US)

(21) Appl. No.: 19/328,981

(22) Filed: Sep. 15, 2025

Related U.S. Application Data
(60) Provisional application No. 63/697,296, filed on Sep. 20, 2024.

(51) Int. Cl. A61M 16/08; A61M 39/20

(52) U.S. Cl. CPC A61M 16/0816; A61M 39/20; A61M 2207/00

(57) ABSTRACT

A medical gas retention system including a nozzle having an inlet, an outlet, and a fluid flow passageway extending from the inlet to the outlet and configured to convey medical gas from the inlet and through the outlet. The medical gas retention system also includes an arm coupled to the nozzle and including an occluding surface. The arm has a first position and a second position different from the first position, wherein the arm is biased toward the second position and the occluding surface automatically closes the outlet when the arm is in the second position to prevent medical gas flow through the outlet.

### CROSS-REFERENCE TO RELATED APPLICATION

This application claims priority to and the benefit of U.S. Provisional Application Ser. No. 63/697,296, filed on Sep. 20, 2024, entitled “Medical Gas Retention System.” The entire content of the application is incorporated herein by reference in its entirety.

### FIELD

The present disclosure relates to fluid systems and valves, and, more particularly, to medical gas retention systems.

### BACKGROUND

Pressurized fluid systems often include valves to retain pressurized fluid. In some examples, the valve is disposed near or on an outlet of a pressurized tube or piping system. Retaining the pressurized fluid preserves valuable resources. In some examples, the pressurized fluid is a gas, such as oxygen, or a liquid.

Specifically, hospitals and other medical facilities include pressurized oxygen systems for administration to patients. In such systems, medical professionals connect a patient's tubing to the pressurized oxygen system and open a valve to deliver pressurized air or oxygen to the patient. In this example, the oxygen provided to the patient is typically either generated onsite via an oxygen concentrator or generated offsite and transported via pressurized cylinders.

In one example, the pressurized oxygen systems in medical facilities (e.g., hospitals) are connected to various breathing apparatus to improve a patient's oxygen saturation. The provision of oxygen is measured and controlled via a flowmeter, such as a Thorpe tube flowmeter, that includes a valve. A medical professional can control the administration of oxygen to a patient by actuating the valve on the flowmeter. The medical professional should close the valve on the flowmeter when disconnecting the breathing apparatus from the flowmeter.

### SUMMARY

Disclosed herein is a medical gas retention system, including: a nozzle having an inlet, an outlet, and a fluid flow passageway extending from the inlet to the outlet and configured to convey medical gas from the inlet and through the outlet. The medical gas retention system also includes an arm coupled to the nozzle and including an occluding surface. The arm has a first position and a second position different from the first position and the arm is biased toward the second position. The occluding surface automatically closes the outlet when the arm is in the second position to prevent medical gas flow through the outlet.

In some variations, the nozzle may include a base disposed about the inlet. The nozzle may also include a bracket coupled to the base, the arm pivotably coupled to the bracket. The bracket may be disposed radially outward from the inlet.

In other variations, the medical gas retention system also includes a hinge, wherein the hinge may pivotably couple the arm to the nozzle. Additionally, the hinge may include a spring to bias the arm towards the second position.

In yet further variations, the arm may include a distal head, the occluding surface disposed on the distal head. Also, the outlet may have a distal surface, the distal surface and the occluding surface may define complementary angles. In such examples, the occluding surface may sealingly close the outlet in the second position.

Also disclosed herein is a medical gas retention system, including: a nozzle having an inlet, an outlet having a distal surface, and a fluid flow passageway extending from the inlet to the outlet and configured to convey medical gas from the inlet and through the outlet. The medical gas retention system also includes an arm coupled to the nozzle and including an occluding surface, the arm having a first position and a second position different from the first position. The distal surface and the occluding surface define complementary angles and the occluding surface closes the outlet when the arm is in the second position.

In some variations, the arm may be biased toward the second position. Additionally, the arm may also include a distal head, and the occluding surface may be disposed on the distal head. The nozzle may also include a base disposed about the inlet. The base may include a bracket, and the arm may be pivotably coupled to the bracket.

In other variations, the medical gas retention system may include a spring coupled to the arm to bias the arm towards the second position. Additionally, the occluding surface may sealingly close the outlet when the arm is in the second position.

Also disclosed herein is a method of assembling a medical gas retention system, including: providing a nozzle having an inlet, an outlet, and a fluid flow passageway extending from the inlet to the outlet and configured to convey fluid from the inlet and through the outlet. The method also includes pivotably coupling an arm to nozzle, wherein the arm has a first position and a second position different from the first position, the arm being biased toward the second position. Additionally, the arm is configured to close the outlet when in the second position.

In some variations, the method may include pivotably coupling the arm to the nozzle includes coupling the arm to a bracket on the nozzle. In some such examples, pivotably coupling the arm to the nozzle may further include coupling a spring to the arm, wherein the spring biases the arm toward the second position. The method may also include aligning a distal surface of the outlet with a sealing surface of the arm.

### BRIEF DESCRIPTION OF DRAWINGS

The present disclosure is described in the following detailed description in conjunction with the drawings, wherein:

FIG. 1 is a block diagram of an example fluid retention assembly in accordance with the present disclosure.

FIG. 2 is a perspective view of a first example medical gas retention system of the present disclosure.

FIG. 3 is a front view of the first example medical gas retention system of FIG. 2.

FIG. 4 is a side view of the first example medical gas retention system of FIG. 2.

FIG. 5 is a cross-sectional view of the first example medical gas retention system of FIG. 2 in a closed configuration taken along the line A-A of FIG. 3.

FIG. 6 is a cross-sectional view of the first example medical gas retention system of FIG. 2 in an open configuration.

FIG. 7 is a perspective view of a second example medical gas retention system, in an open configuration.

FIG. 8 is a perspective view of the second example medical gas retention system of FIG. 7 in a closed configuration.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

The present disclosure is directed to a medical gas retention system to prevent loss of valuable gases administered in medical environments. It is costly to generate and/or store medical gases including: medical air, oxygen, nitrogen, nitrous oxide, carbon dioxide, helium, nitric oxide, and many other gases utilized in medical environments.

More specifically, a medical gas retention system that automatically closes in response to disconnection from a fluid receiver is disclosed. As a result, loss of a valuable, pressurized fluid, such as oxygen, from a pressurized fluid source is minimized. The medical gas retention system includes a nozzle with an arm and an occluding surface configured to close the nozzle. In some examples, the arm is biased such that the arm automatically closes the nozzle when the nozzle is not in use. The arm may be biased by a spring or weighted such that the nozzle is closed and sealed, therein preventing loss of valuable pressurized fluid.

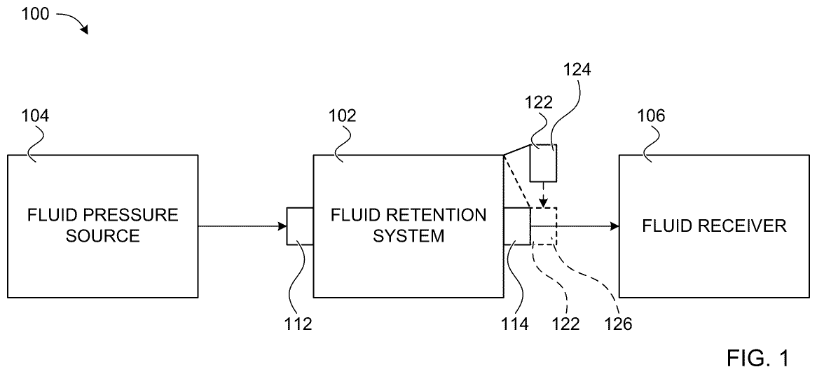

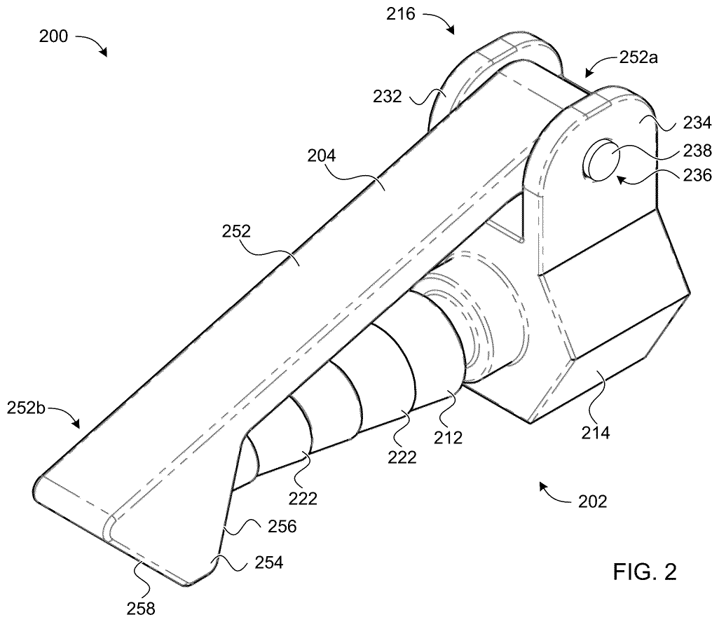

Referring now to FIG. 1, a schematic diagram of the fluid retention assembly 100 utilizing a fluid retention system 102 of the present disclosure (described in greater detail below in connection with FIGS. 2-8) is depicted. The fluid retention assembly 100 includes a fluid pressure source 104, the fluid retention system 102, and a fluid receiver 106, such as during operation of the fluid retention assembly 100. The fluid pressure source 104 is fluidly coupled to an inlet 112 of the fluid retention system 102, and the fluid receiver 106 is fluidly coupled to an outlet 114 of the fluid retention system 102. Further, the fluid retention system 102 includes an occlusion surface 122 having a first position 124 in which the occlusion surface 122 is not in contact with the outlet 114 of the fluid retention system 102, and a second position 126 in which is the occlusion surface 122 is in contact with the outlet 114, as explained more below. In the first position 124, the outlet 114 of the fluid retention system 102 may be coupled to the fluid receiver 106. However, when the fluid receiver 106 is disconnected from the fluid retention system 102, the occlusion surface 122 moves from the first position 124 to the second position 126 to close the outlet 114 of the fluid retention system 102. More specifically, and as discussed more below, the occlusion surface 122 may be biased toward the second position 126 such that the outlet 114 is automatically closed when disconnected from the fluid receiver 106.

In the present example, the fluid pressure source 104 stores or provides a fluid (i.e., liquid or gas, such as a medical gas, including oxygen) at a pressure greater than ambient conditions or greater than the outlet 114 of the fluid retention system 102, for example. In some examples, the fluid pressure source 104 includes a pressurized tank or pump coupled to an unpressurized tank of liquid or gas. In a hospital environment, the pressure source 104 may include an oxygen concentrator and pump or a pressurized oxygen tank, in one example. It will be appreciated that other pressure sources 104 may alternatively and/or additionally be used and still fall within the scope of the present disclosure.

The fluid receiver 106 is configured to receive the fluid, such as pressurized fluid, from the fluid pressure source 104. The fluid receiver 106 may include tubing configured for administering the fluid to a patient. For example, the fluid receiver 106 could include a nasal cannula for administering pressurized oxygen to the patient. It will be appreciated that various other devices for administering pressurized oxygen to the patient known to persons having ordinary skill in the art may additionally and/or alternatively be used and still fall within the scope of the present disclosure.

In the fluid retention assembly 100 shown in FIG. 1, the fluid pressure source 104 can be fluidly coupled to the fluid retention system 102 via one or more fluid passageways. For example, the fluid retention assembly 100 may include rigid and/or flexible conduit, piping, and/or tubing fluidly coupling the pressure source 104 to the fluid retention system 102. Additionally, and as will be appreciated, the fluid passageways may include various couplings, valves, sensors, or other elements normally incorporated in pressurized fluid passageways. Similarly, the fluid receiver 106 may likewise be fluidly coupled to the fluid retention system 102 via one or more fluid passageways, similar or identical to the fluid passageways coupling the pressure source 104 and the fluid retention system 102.

Referring now to FIGS. 2, 3, and 4, a medical gas retention system 200 according to the present disclosure and a first example of the fluid retention system 102 of FIG. 1 is depicted. In various examples, the medical gas retention system 200 is optimized to retain medical gases (e.g., medical air, oxygen, nitrogen, nitrous oxide, carbon dioxide, helium, nitric oxide), but could be applicable to a wider variety of fluids in some examples. The medical gas retention system 200 includes a nozzle 202 configured to convey medical gas through the nozzle 202, as explained more below, and an arm 204 coupled to the nozzle 202, such as pivotably coupled to the nozzle 202. In the present example, the nozzle 202 is configured for use in a healthcare facility. The nozzle 202 includes a pipe 212, a base 214, and a bracket 216 coupled to the base 214. The arm 204 can also be indirectly coupled to the nozzle 202, such as the arm 204 being pivotably coupled to the bracket 216. In other examples, the nozzle 202 and arm 204 can be differently arranged relative to each other. In the present example, the medical gas retention system 200 is made of a material that can be sterilized for a healthcare environment (e.g., stainless steel, polymers), but may alternatively be made of any material and still fall within the scope of the present disclosure.

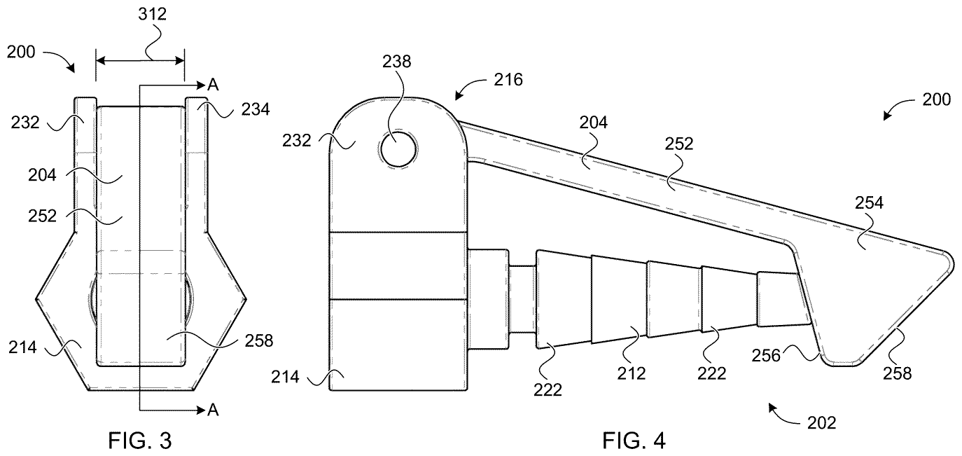

The nozzle 202 includes the pipe 212 having a generally frustoconical shape with a plurality of serrated, frustoconical shoulders 222. The frustoconical shoulders 222 are configured to couple to fluid passageways (e.g., tubing) having a wide variety of internal diameters. In the present example, the pipe 212 is integrally formed with the base 214. In other examples, the base 214 may be coupled to the nozzle 202 via welding, adhesive, or other fastening means. The pipe 212 is generally circular when viewed from the front (e.g., from the perspective view shown in FIG. 3)

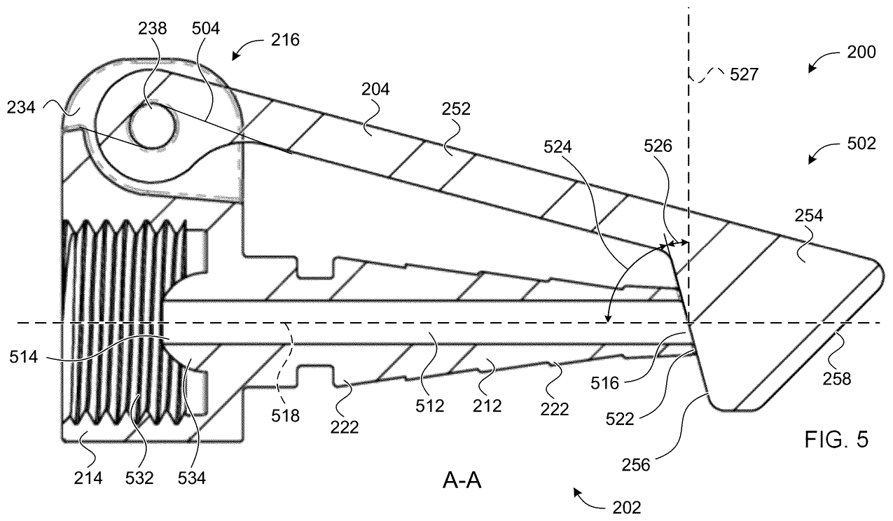

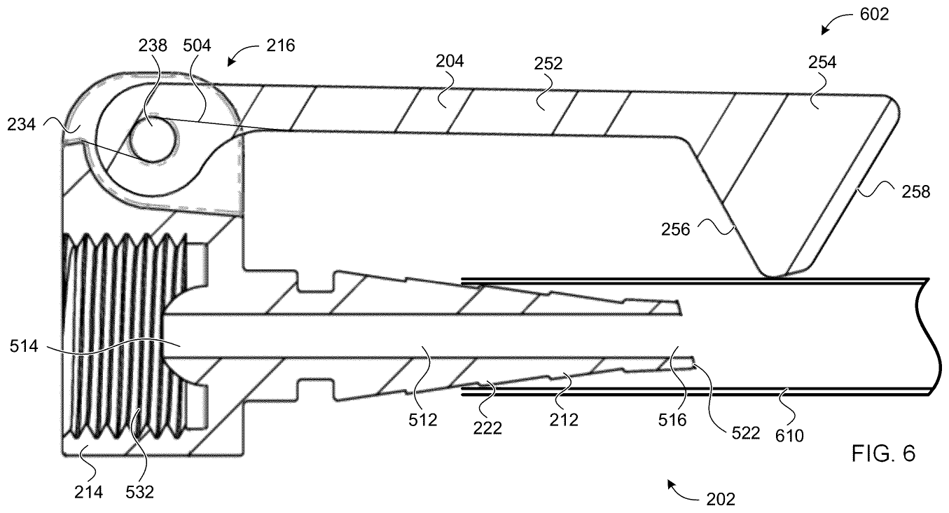

The nozzle 202 further includes the bracket 216. As shown in FIG. 2, in this example, the bracket 216 is coupled to the base 214. The bracket 216 is generally U-shaped and includes a first tab 232 and a second tab 234 spaced apart from the first tab 232. As shown in FIG. 3, the arm 204 has a width 312 approximately equal to the distance between the first tab 232 and the second tab 234. The first and second tabs 232, 234 each include an aperture 236, such that the aperture 236 of the first tab 232 is aligned with the aperture 236 of the second tab 234. The medical gas retention system 200 also includes a hinge 238, and the aligned apertures 236 are each configured to receive the hinge 238 (e.g., an axle). As shown in FIGS. 2, 3, and 4, the first and second tabs 232, 234 are in a plane perpendicular to a plane of the pipe 212, and the hinge 238 defines an axis perpendicular to the both the bracket 216 (e.g., the planes of the first and second tabs 232, 234) and the plane of the pipe 212 defined by the cross-sectional view A-A (shown in FIG. 5). The hinge 238 pivotably couples the arm 204 to the nozzle 202 and/or the bracket 216. In some examples, the hinge 238 includes a spring 504 (shown in FIGS. 5 and 6) to bias the arm 204 toward a second position (discussed in greater detail in connection with FIGS. 5 and 6).

The arm 204 includes a strut 252 having a proximal end 252a and a distal end 252b (FIG. 2) with a distal head 254. The arm 204 also includes an occluding surface 256 that is disposed on the distal head 254. The distal head 254 further includes a control surface 258 disposed opposite the occluding surface 256. As will be described in greater detail in connection with FIGS. 5 and 6, the occluding surface 256 closes the outlet of the nozzle 202, and the control surface 258 facilitates actuating the arm 204 against the bias of the arm 204 (discussed in greater detail in connection with FIG. 6). In the present example, the arm 204 is rigid, but, in other examples, part or all of the arm 204 may be flexible or semi-flexible. Additionally, in the present example, the strut 252 and the distal head 254 are integrally formed, but may alternatively be coupled together.

Referring now to FIGS. 5 and 6, FIG. 5 is a cross-sectional view of the medical gas retention system 200 taken along the line A-A of FIG. 3 with the nozzle 202 in a closed configuration. FIG. 6 is a cross-sectional view of the medical gas retention system 200 with the nozzle 202 in an open configuration. More specifically, the arm 204 has a second position 502, shown in FIG. 5, with the occluding surface 256 closing the nozzle 202 such that the nozzle is in the closed configuration. The arm 204 has a first position 602, shown in FIG. 6, different from the second position 502. In the first position 602, the nozzle 202 is in an open configuration. The second position 502 is different from the first position 602. As described above, the arm 204 is pivotable between at least the first position 602 and the second position 502. In the present example, the arm 204 is biased toward the second position 502 and the occluding surface 256 automatically closes the outlet 516 of the nozzle 202 when the arm 204 is in the second position 502 to prevent medical gas flow through nozzle 202.

As shown in FIG. 5, the nozzle 202 has a fluid flow passageway 512, an inlet 514 and an outlet 516. The fluid flow passageway 512 extends from the inlet 514 to the outlet 516 and is configured to convey medical gas from the inlet 514 to the outlet 516 and through the outlet 516. In preferred examples, the fluid flow passageway 512 is sized to provide adequate medical gas flow from the inlet 514 to the outlet 516. In the present example, the inlet 514 and the outlet 516 are coaxial and disposed along a longitudinal axis 518. However, in other examples, the inlet 514 and the outlet 516 could be non-coaxial (e.g., the fluid flow passageway 512 is not linear). The inlet 514 is disposed within the base 214 and the outlet 516 is disposed opposite the inlet 514.

As shown in FIG. 5, the nozzle 202 includes the base 214 disposed about the inlet 514. As a result, the bracket 216 is disposed radially outward from the inlet 514. In other examples, the bracket 216 and the inlet 514 could be disposed elsewhere on the nozzle 202. For example, the bracket 216 could be disposed between the inlet 514 and the outlet 516. Alternatively, the bracket 216 could be disposed upstream of the inlet 514.

As further depicted in FIG. 5, the outlet 516 has a distal surface 522. In the present example, the distal surface 522 is angled relative to the longitudinal axis 518. Similarly, the occluding surface 256 is also angled. Angle 524 defines the angle between the longitudinal axis 518 and the distal surface 522. Additionally, angle 526 defines the angle between the occluding surface 256 and a plane 527 perpendicular to the longitudinal axis 518. In the present example, the angle 524 and the angle 526 are complementary angles, that is, the sum of the angles 524, 526 is ninety degrees (90°). As a result, the distal surface 522 and the occluding surface 256 define the complementary angles 254, 256. In other examples, the angles 524, 526 are not complementary, but the occluding surface 256 still closes the outlet 516 in the second position 502 because the occluding surface 256 is flush with the distal surface 522 of the outlet 516 when in the second position 502.

As shown in FIG. 5, the occluding surface 256 closes the outlet 516 when the arm 204 is in the second position 502. In some examples, the occluding surface 256 sealingly closes the outlet 516 when the arm 204 is in the second position 502. As a result, the occluding surface 256 prevents pressurized medical gas or reduces the amount of pressurized medical gas (e.g., from a fluid pressure source 104) from passing through the outlet 516.

Further, as shown in FIG. 5, the base 214 includes a coupling mechanism 532. In the present example, the coupling mechanism 532 is a threaded connection. But in other examples, the coupling mechanism 532 could be any coupling mechanism for coupling the base to a pressure source (e.g., a Thorpe tube flowmeter fluidly coupled to an oxygen concentrator or pressurized oxygen tank). Further, the inlet 514 includes a rounded portion 534 extending partially into the base 214, such that the 514 may be semi-spherical in shape in one example. In the present example, the semi-spherical shape facilitates sealing with a pressure source (e.g., Thorpe tube flowmeter). However, in various other examples, the rounded portion 534 could be any shape and still fall within the scope of the present disclosure.

Referring now to FIG. 6, the medical gas retention system 200 is depicted with the arm 204 in a first position 602, and the nozzle 202 is coupled to tubing 610. In some examples, the tubing 610 corresponds to the fluid receiver 106 or the tubing 610 is coupled to the fluid receiver 106. In a medical facility, the tubing 610 can be coupled to a nasal cannula or other breathing aid device, for example. It will be appreciated that the tubing 610 may alternatively and/or additionally be coupled to any other known device and still fall within the scope of the present disclosure. As shown in FIG. 6, the tubing 610 engages one of the frustoconical shoulders 222 to selectively secure the tubing 610 on the nozzle 202. In various examples, the tubing 610 can be selectively secured to the nozzle 202 in any other manner.

As shown in FIG. 6, the arm 204 is pivotably biased toward the second position 502 and rests against the tube 610. In some examples, the hinge 238 can include the spring 504 (e.g., a torsional spring, extension/compression spring, spiral spring). In some examples, the spring 504 is coupled, at least on one end, to the arm 204. As discussed above, the hinge 238 can include the spring 504 to bias the arm 204 toward the second position 502. In such examples, the spring 504 acts upon the hinge 238 and/or the arm 204. In other examples, the arm 204 and distal head 254 are sufficiently weighted to bias the arm 204 to the second position 502 and close the outlet 516 due to gravity.

When the tube 610 is uncoupled from the nozzle 202, the arm 204 automatically transitions to the second position 502. As a result, the occluding surface 256, and by extension the arm 204, is configured to automatically close the nozzle 202. In various examples, the occluding surface 256 automatically sealingly closes the outlet 516 of the fluid passageway 512 when the arm 204 automatically transitions to the second position 502 from the first position 602.

Additionally, when a medical practitioner needs to couple the tube 610 to the nozzle 202, the control surface 258 can be engaged to move the arm 204 to the first position 602. Specifically, the medical practitioner can push up on the control surface 258 with a finger or knuckle while holding the tube 610 so that the medical practitioner can couple the tube 610 to the nozzle 202 with one hand. In other examples, the medical practitioner can lift the arm 204 up with one hand and couple the tube 610 to the nozzle 202 with the other hand.

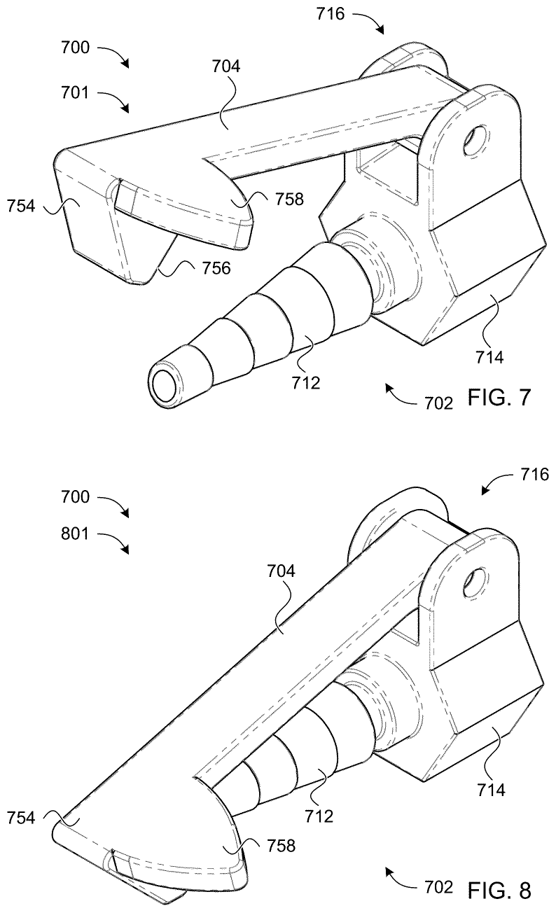

FIGS. 7 and 8 illustrate an alternative medical gas retention system 700 and a second example of the fluid retention system 102 of FIG. 1. As shown in FIGS. 7 and 8, the medical gas retention system 700 is substantially similar to the medical gas retention system 200. For example, the medical gas retention system 700 includes a nozzle 702 and an arm 704. The nozzle 702 is generally identical to the nozzle 202 and includes pipe 712, base 714, and bracket 716 that are identical to the nozzle 202. The arm 704 is similar to the arm 204, but unlike the arm 204 of the medical gas retention system 200, the arm 704 includes a distal head 754 with a projecting tab 758. In various examples, the medical gas retention system 700 is optimized to retain medical gases (e.g., oxygen, medical air), but could be applicable to a wider variety of fluids.

The projecting tab 758 extends from the distal head 754. The projecting tab 758 provides a surface that is readily identifiable by medical professionals for actuating the arm 704 of the medical gas retention system 700. Further, the projecting tab 758 moves the control surface for the arm 704 away from the nozzle 712. This separation reduces the likelihood that the hand of the medical professional will dirty the nozzle 712 or limit access to the nozzle 712 when attempting to couple tubing to the nozzle 712. As shown in FIGS. 7 and 8, the projecting tab 758 is approximately triangular in shape, but in various other examples, the projecting tab 758 can be semi-circular, rectangular, ogive, pentagonal, or any other shape and still fall within the scope of the present disclosure. Additionally, in the present example, the projecting tab 758 is integrally formed with the arm 704 and distal head 754, but in other examples, the projecting tab 758 can be coupled to the distal head 754 after the arm 704 is manufactured and again still fall within the scope of the present disclosure.

The medical gas retention system 700 operates identically to the medical gas retention system 200. For example, the arm 704 is pivotable relative to the nozzle 202 between a first position 701 and a second position 801. Similar to the arm 204, the arm 704 is biased toward the second position 801 and the occlusion surface 756 automatically closes the nozzle 702 as discussed above with respect to arm 204.

The medical gas retention systems 200, 700 discussed above can be manufactured or modified from an existing nozzle. Specifically, a method of assembling the medical gas retention system 200 includes providing the nozzle 202 having the inlet 514, the outlet 516, and the fluid flow passageway 512 extending from the inlet 514 to the outlet 516 and configured to convey medical gas from the inlet 514 and through the outlet 516. The method also includes pivotably coupling the arm 204 to the nozzle 202, wherein the arm 204 has the first position 602 and the second position 502 different from the first position 602, the arm 204 being biased toward the second position 502. The arm 204 is configured to close the outlet 516 when in the second position 502.

In one example, pivotably coupling the arm 204 to the nozzle 202 may include coupling the arm 204 to a bracket 216 on the nozzle 202. Additionally, and in another example, pivotably coupling the arm 204 to the nozzle 202 may include coupling a spring 504 to the arm 204 and/or the hinge 238. In such examples, the spring 504 can bias the arm 204 toward the second position 502.

Assembling the medical gas retention system 200 includes configuring the arm 204 such that the arm closes the outlet 516 when the arm 204 is in the second position 502. Configuring the arm 204 to close the outlet 516 includes aligning a distal surface 522 of the outlet 516 with an occluding surface 256 of the arm 204.

The medical gas retention systems 200, 700, as described herein, provide several benefits over other seal components. First, the medical gas retention systems 200, 700 provide an automatic closure of the nozzle 202, 702 when the outlet 516 of the nozzle 202, 702 is uncoupled from a fluid receiver (e.g., fluid receiver 106). Second, the medical gas retention system 200, 700 is configured for straightforward, easy use in busy, hectic environments, such as medical environments. For example, the automatic closure by the arm 204, 704 can be easily overcome by a medical practitioner needing to quickly couple a fluid receiver (e.g., fluid receiver 106).

Those skilled in the art will recognize that a wide variety of modifications, alterations, and combinations can be made with respect to the above described examples without departing from the spirit and scope of the invention(s) disclosed herein, and that such modifications, alterations, and combinations are to be viewed as being within the ambit of the inventive concept(s).

What is claimed is:

1. A medical gas retention system, comprising:
a nozzle having an inlet, an outlet, and a fluid flow passageway extending from the inlet to the outlet and configured to convey medical gas from the inlet and through the outlet; and
an arm coupled to the nozzle and including an occluding surface, the arm having a first position and a second position different from the first position, wherein the arm is biased toward the second position and the occluding surface automatically closes the outlet when the arm is in the second position to prevent medical gas flow through the outlet.

2. The system of claim 1, the nozzle further comprising a base disposed about the inlet.

3. The system of claim 2, wherein the nozzle further includes a bracket coupled to the base, the arm pivotably coupled to the bracket, and the bracket is disposed radially outward from the inlet.

4. The system of claim 1, wherein the medical gas includes at least one of oxygen, nitrogen, nitrous oxide, carbon dioxide, helium, and nitric oxide.

5. The system of claim 1, further comprising a hinge, wherein the hinge pivotably couples the arm to the nozzle.

6. The system of claim 5, wherein the hinge includes a spring to bias the arm towards the second position.

7. The system of claim 1, the arm further including a distal head, the occluding surface disposed on the distal head.

8. The system of claim 1, wherein the outlet has a distal surface, the distal surface and the occluding surface define complementary angles.

9. The system of claim 8, wherein the occluding surface sealingly closes the outlet when the arm is in the second position.

10. A medical gas retention system, comprising:
a nozzle having an inlet, an outlet having a distal surface, and a fluid flow passageway extending from the inlet to the outlet and configured to convey medical gas from the inlet and through the outlet; and
an arm coupled to the nozzle and including an occluding surface, the arm having a first position and a second position different from the first position;
wherein the distal surface and the occluding surface define complementary angles and the occluding surface closes the outlet when the arm is in the second position.

11. The system of claim 10, wherein the arm is biased toward the second position.

12. The system of claim 11, the arm further including a distal head, the occluding surface disposed on the distal head.

13. The system of claim 10, the nozzle further comprising a base disposed about the inlet, and wherein the base further incudes a bracket, the arm pivotably coupled to the bracket.

14. The system of claim 10, wherein the medical gas includes at least one of oxygen, nitrogen, nitrous oxide, carbon dioxide, helium, and nitric oxide.

15. The system of claim 13, further comprising a spring coupled to the arm to bias the arm towards the second position.

16. The system of claim 10, wherein the occluding surface sealingly closes the outlet when the arm is in the second position.

17. A method of assembling a medical gas retention system, comprising:
providing a nozzle having an inlet, an outlet, and a fluid flow passageway extending from the inlet to the outlet and configured to convey fluid from the inlet and through the outlet; and
pivotably coupling an arm to nozzle, wherein the arm has a first position and a second position different from the first position, the arm being biased toward the second position;
wherein the arm is configured to close the outlet when in the second position.

18. The method of claim 17, wherein pivotably coupling the arm to the nozzle includes coupling the arm to a bracket on the nozzle.

19. The method of claim 17, wherein pivotably coupling the arm to the nozzle further includes coupling a spring to the arm, wherein the spring biases the arm toward the second position.

20. The method of claim 17, further comprising aligning a distal surface of the outlet with an occluding surface of the arm.
